(19) United States
(12) Patent Application Publication
(10) Pub. No.: US 2004/0101495 A1
(43) Pub. Date: May 27, 2004

(54) Method for improving dental health

(76) Inventors: Leena Nase, Tampere (FI); Jukka Meurman, Espoo (FI); Maija Saxelin, Vantaa (FI)

Correspondence Address: NIXON & VANDERHYE, PC, 1100 N GLEBE ROAD, 8TH FLOOR, ARLINGTON, VA, 22201-4714, US

(21) Appl. No.: 10/305,355

(22) Filed: Nov. 27, 2002

(51) Int. Cl. A61K 7/28; A61K 47/00

(52) U.S. Cl. 424/50; 424/93.45; 424/439

(57) ABSTRACT

The invention relates to methods for improving oral health. In particular, methods for improving oral health and dental health and for reducing the risk or caries by utilization of probiotic bacteria are described.

### FIELD OF THE INVENTION

The invention relates to methods for improving oral health, In particular, methods for improving oral health and dental health and for reducing the risk or caries by utilization of probiotic bacteria are described.

### BACKGROUND OF THE INVENTION

Oral health and dental health are very complex issues in which a multitude of different factors are involved, one of the most important being the microbial flora in the oral cavity, the mucosa and on the teeth.

Also caries risk assessment is a complex issue. Researchers have been looking for the factors that would enable to predict the development of caries lesions. Many variables have been investigated for their association with the carious process. Risk indicators may include socio-economic factors such as income, psychosocial factors such as health attitudes, oral health habits, clinical variables such as the number of filled teeth, microbiological parameters such as mutans streptococcus and lactobacillus counts, and salivary calcium content (Hausen et al. Can caries be predicted? In Thylstrup A, Fejerskoj O (eds): Textbook of Clinical Cariology. Copenhagen, Munksgaard, ed 2, 1996, pp-393-411).

Streptococci and lactobacilli, the most abundant of the acidogenic species resident in the oral cavity, are recognised as associated with the presence and onset of dental caries. (Loesche, W J: Role of Streptococcus mutans in human dental decay. Microbiol Rev 1986; 50:353-380). In general. lactobacilli colonize acidic environments such as fissures and interdental spaces, and produce acids from fermentable sugars. Mutans streptococci and lactobacilli are often found together in carious lesions (Babaahmady et al,: Ecological study of Streptococcus mutans, Streptococcus sobrinus and Lactobacillus spp. at subsites from approximal dental plaque from children. Caries Res 1998;32:51-58). Caries is also known to correlate well with the number of oral lactobacilli in children (Granath et al.: Salivary lactobacilli explain dental caries better than salivary mutans streptococci in 4-5-year-old children. Scand J Dent Research 1994;102:319-323). Heterofermentative lactobacilli have been shown to increase caries activity in monkeys (Bowen W H: The lactobacilli in the saliva, plaque and carious dentine in Macaca irus. J Path Bacteriol 1967;94:55-61).

A saliva sample positive for S. mutans correlates with clinical findings on caries (Splieth and Bernhardt. Prediction of caries development for molar fissures with semiquantitative mutans streptococci test. Eur J Oral Sci 1999;107:164-169). Caries experience and/or mutans streptococcal levels in primary dentition may also be indicators of caries in permanent dentition (al-Shalan et al.: Primary incisor decay before age 4 as a risk factor for future dental caries. Pediatr Dent 1997;19:3741). Plaque is an indicator of caries risk in 3-year-old children and therefore should be a key factor in health education (Mattila et al.: Changes in dental health and dental health habits from 3 to 5 years of age. J Public Health Dent 1998:58:270-274). A pooled plaque and saliva sample produces more exact data about oral cariogenic bacteria than the mutans streptococcal level in saliva alone. It has also been shown that mutans streptococcal clones may selectively colonize specific hard-tissue sites (Grönroos and Alaluusua: Site-specific oral colonization of mutans streptococci detected by arbitrarily primed PCR fingerprining. Caries Res 2000;34:474-480).

### SHORT DESCRIPTION OF THE INVENTION

The object of the present invention is to provide a method for improving the oral health of human beings and animals.

It is a further object of the present invention to provide a method for improving the dental health of human beings and animals.

It is a further object of the present invention to provide a method for preventing and effectively reducing the risk of caries.

The objects of the present invention are achieved by a method comprising administering to an individual Lactobacillus rhamnosus GG, ATCC 53103.

The present invention thus relates to a method for improving the oral health in a subject, the method comprising administering to an individual in need of such treatment Lactobacillus rhamnosus GG, ATCC 53103, in an amount sufficient to achieve the desired result.

The present invention further relates to a method for improving the dental health in a subject, the method comprising administering to an individual in need of such treatment Lactobacillus rhamnosus GG, ATCC 53103, in an amount sufficient to achieve the desired result.

The present invention also relates to a method for preventing and reducing the risk of caries in a subject, the method comprising administering to an individual in need of such treatment Lactobacillus rhamnosus GG, ATCC 53103, in an amount sufficient to achieve the desired result.

The method covers all aspects of preventing and reducing the risk of caries, such as preventing the development of caries, prophylaxis and treatment of caries and caries induction, and alleviating the first signs of caries.

The invention further relates to improving the beneficial effects of Lactobacillus rhamnosus GG, ATCC 53103, in methods involving improving the oral health, improving the dental health, and preventing and reducing the risk of caries in a subject, the methods comprising administering to an individual in need of such treatment a calcium compound and Lactobacillus rhamnosus GG, ATCC 53103, in amounts sufficient to achieve the desired result.

Preferably, the calcium compound is a soluble calcium salt such as calcium lactate, gluconate, phosphate, carbonate, citrate, malate, hydroxide, chloride or formate.

The invention also relates to a method for improving oral health, improving dental health, or preventing and reducing the risk of caries in a subject, the method comprising administering to an individual in need of such treatment Lactobacillus rhamnosus GG, ATCC 53103, as such in an amount sufficient to achieve the desired result.

The invention also relates to a method for improving oral health, improving dental health, or preventing and reducing the risk of caries in a subject, the method comprising administering to an individual in need of such treatment Lactobacillus rhamnosus GG, ATCC 53103, in a physiologically acceptable vehicle and in an amount sufficient to achieve the desired result.

Preferably, the vehicle is milk or a milk product.

### DESCRIPTION OF THE DRAWING

FIG. 1 shows the effects of LGG milk (a) and control milk (b) on caries risk in day-care children in three age groups, at baseline and at the end.

### DETAILED DESCRIPTION OF THE INVENTION

The present invention resides in the beneficial effects of Lactobacillus rhamnosus GG, ATCC 53103 (LGG) in improving oral health and dental health and in preventing and significantly reducing the risk of caries in human beings and animals.

In connection with the present invention, the beneficial effects can be further improved by the combined use of Lactobacillus rhamnosus GG, ATCC 53103 (LGG), and agents or vehicles enhancing the effects of said microorganism or exerting beneficial effects of their own.

As a preferred compound, the present invention involves the use of a calcium compound, preferably a calcium salt.

As vehicle, the present invention in a preferred embodiment involves the use of a vehicle comprising such a calcium compound. Preferably, said vehicle is milk.

Lactobacillus rhamnosus GG (LGG) has been described in the prior art. It is, for example, the subject matter of U.S. Pat. No. 5,032,399, Gorbach & Goldin. The strain is isolated from human faeces, it is able to grow well in pH 3 and survives even lower pH values as well as high bile acid contents. The strain exhibits excellent adhesion to both mucus and epithelial cells. Lactic acid yield from glucose is good: when grown in MRS broth, the strain produces 1.5-2% of lactic acid. The strain does not ferment lactose and thus it does not produce lactic acid from lactose. The strain employs the following carbohydrates. D-arabinose, ribose, galactose, D-glucose, D-fructose, D-mannose, rhamnose, dulcitol, inositol, mannitol, sorbitol, N-acetylglucosamine, amygdalin, arbutin, esculin, salicin, cellobiose, maltose, saccharose, trehalose, melezitose, gentibiose, D-tagatose, L-fucose, and gluconate The strain grows well at +15-45° C., the optimum temperature being 30-37° C. Lactobacillus rhamnosus GG is deposited with the depository authority American Type Culture Collection under accession number ATCC 53103.

Lactobacillus rhamnosus GG ferments sucrose very slowly, and cannot ferment lactose. In general, the beneficial effects of LGG in the human body are well documented, and it is used particularly in stabilizing the gastrointestinal microflora (Gorbach S L: Lactic acid bacteria and human health. Ann Med 1990;22:37-41; Fuller R: Probiotics in human medicine. Gut 1991;32:439-442; Salminen et al.: Lactic acid bacteria in health and disease; in Salminen S, von Wright A (eds): Lactic acid bacteria, New York, Dekker, 1993; pp.199-225; Perdigon et al.: Symposum: Probiotic bacteria for humans: Clinical systems for evaluation of effectiveness. Immune system stimulation by probiotics. J Dairy Sci 1995;78:1597-1606; Wagner et al.: Biotherapeutic effects of probiotic bacteria on candidiasis in immunodeficient mice. Infect Immun 1997;65:4165-4172; Miettinen et al., 1998; Pelto et al., 1998; Shida et al., 1998; Dunne et al., 1999, Gorbach, 2000; Guandalini et al., 2000). Lactobacillus rhamnosus GG is nowadays widely used in dairy products in many countries.

Lactobacillus rhamnosus GG, ATCC 53103 (LGG), isolated from healthy humans, has been shown to produce a substance with potent inhibitory activity on a wide range of bacterial species including Streptococcus spp. (Silva et al.: Antimicrobial substance from a human Lactobacillus strain. Antimicrob Agents Chemother 1987;31:1231-1233). LGG has also been shown to temporarily colonize the mouth and to produce a substance which has a weak inhibitory effect in vitro on a caries pathogen, Streptococcus sobrinus (Meurman et al.: Recovery of Lactobacillus strain GG (ATCC 53103) from saliva of healthy volunteers after consumption of yoghurt prepared with the bacterium. Microbiol Ecol Health Dis 1994;7:295-298; Meurman et al.: Effect of Lactobacillus rhamnosus strain GG (ATCC 53103) on the growth of Streptococcus sobrinus in vitro. Eur J Oral Sci 1995;103:253-258). The latter document describes the isolation of a substance, the chemical structure of which is not further evaluated, but which is shown to possess a weak in vitro growth inhibition activity on one particular bacterium, Streptococcus sobrinus. The effect was restricted to low pH values of below 5, and was observed only during cultivation in one particular medium, MRS broth. It has also been reported that Lactobacillus rhamnosus strain GG cannot colonize tooth surfaces (Busscher et al.: In vitro adhesion to enamel and in vivo colonization of tooth surfaces to lactobacilli from bio-yoghurt. Caries Res 1999;33:403404).

For use in accordance with the objects of the present invention, the microorganism is cultivated using processes conventional in the art. It can be cultivated as a pure culture or in the form of different mixed cultures. The cultures can be used as such or they may be processed as desired for example by purifying, concentrating, lyophilising or finishing them to produce various preparations. One way to produce the bacterium is described in greater detail in example 1; other processes are described for instance in the patent publications FI 92498 and FI20010157, and in Saxelin et al.: Dose response colonisation of faeces after oral administration of Lactobacillus casei strain GG. Microb Ecol Health Dis 1991;4:209-214.

In accordance with the invention, Lactobacillus rhamnosus LGG can be administered as such or in the form of capsules, pills or tablets, for example, manufactured in conventional processes of preparing pharmaceutical products. The microorganism may also be added to diverse edible products, such as foodstuffs, products of the beverage or confectionery industry, health-promoting products, natural products, etc. In the context of the present invention, dairy products, particularly yogurts and other fermented milk products; cheeses and spreads: children's food; and capsules are preferred. A product in the form of a capsule usually only contains a lyophilized culture of the microorganism with a cryoprotective agent.

The end products are prepared in conventional processes, the microorganism being added either in connection with the preparation or thereafter, during the finishing of the end product.

A preferred way of administering the microorganism is to combine it with agents or vehicles enhancing the effects of said microorganism or exerting beneficial effects of their own, For such combined use, the microorganism and the agent or vehicle can be administered in a combined dose form, or they can be administered separately, but in essence simultaneously.

Calcium is the principal mineral in bones and teeth, and developing tooth tissues are affected mainly by factors associated with the intake and metabolism of calcium and phosphorus. Milk and other dairy products contain calcium. Milk on its own, as a complex colloidal fluid of organic and inorganic compounds, seems to be enamel-protective (Gedalia et al.: Enamel softening with Coca Cola and rehardening with milk or saliva. Am J Dent 1991;4:12-22.). Milk also has been reported to have ‘cariostatic properties when ingested at the same time as a cariogenic challenge’ (Bowen and Pearson: Effect of milk on cariogenesis. Caries Res 1993;27:461-466). Calcium lactate, also present in dairy products, has been shown to be anticariogenic (Kashket and Yaskell: Effectiveness of calcium lactate added to food in reducing intraoral demineralization of enamel. Caries Res 1997;31:429-433). In connection with the present invention, milk is, due to both its high calcium content and its documented buffering effects, regarded as a preferred vehicle for administering the microorganism Lactobacillus rhamnosus LGG. It is regarded as especially preferred when the target group is children.

For use in accordance with the objects of the present invention, the microorganism is administered in a sufficient amount to produce the desired effect. This amount will vary depending on the oral and dental health of the subject, as well as of other factors, such as for instance the age, weight and other physiological and physical properties of the subject, the administration form of the microorganism etc. The appropriate dose can be easily determined by a dentist or physician skilled in the art upon the publication of the present invention.

Example 2 describes a study that was designed to assess whether the probiotic LGG bacterium might have beneficial effects in the oral cavity and be used for long-term caries prevention in human. This study was the first clinical long-term trial on the effects of a probiotic bacterium on dental caries and caries risk. By administering LGG in cow's milk it was shown that LGG could affect oral microbiota in a positive way and that children receiving this bacterium in milk developed less caries than children receiving normal milk. The results clearly show that LGG affects the development of caries and caries in human. Naturally, the invention functions equally well as treatment for animal, such as pet animals and domestic animals. As non-restrictive examples cats and dogs may be mentioned,

Contrary to previous reports, in this study, occlusal, approximal and smooth-surface caries did not increase, even though lactobacilli were given to the children. The results thus show the inhibiting effect of LGG on mutans streptococci and other lactobacilli.

The 7-month intervention time was, however, short for caries to progress. In industrialized countries, where fluoride prophylaxis is commonly employed, caries progression is slow. Also, the number of mutans streptococci varies under different biochemical conditions (Bowden and Hamilton: Survival of oral bacteria. Crit Rev Oral Biol Med 1998;9:54-85). A change in the number of initial caries lesions of the 3- to 4-year-old children in the LGG group could indeed be due to an effect on S. mutans, because initial caries is known to correlate with mutans streptococcus counts (Babaahmady et al., 1998). Dairy products such as milk and cheese have been shown to be anticariogenic in humans by increasing the calcium content in plaque (Gedalia et al., 1991; Moynihan et al.: The cariostatic potential of cheese: Cooked cheese-containing meals increase plaque calcium concentration, Br Dent J 1999;187:664-667), but still, a clear difference was observed in caries development and caries risk calculations in the present study where both the test and control children drank milk. Thus the effect must have been due to the intervention of LGG and the unmodified milk could be considered as placebo in this respect.

LGG may also compete with other oral microorganisms by producing antimicrobial substances (Silva et al., 1987), such as pyroglutamic acid (Huttunen et al.: Purification and identification of antimicrobial substances produced by two Lactobacillus casei strains. Dairy J 1995;5:503). Mice that were infected with lactobacilli, but were free from streptococci and enterococci had a lower incidence of colonization by S. gordonii, according to Loach et al. (Colonization of the murine oral cavity by Streptococcus gordonii. Infect Immun 1994;62:2129-2131). LGG has also been shown to increase humoral immunity (Perdigon et al., 1995), and antibodies against it have been detected in the saliva of experimental animals (Negretti et al.: Researches on the intestinal and systemic immunoresponses after oral treatments with Lactobacillus GG in rabbit. Dev Physiopathol and Clin 1997;7:15-21). Consequently, the mechanisms of action of a probiotic bacterium may be manifold also in the oral cavity.

LGG belongs to the homofermentative lactobacilli, and cannot ferment sucrose or lactose. Sucrose is essential in the pathogenesis of dental caries, and hence LGG can be regarded as safe with respect to the teeth, It is also different from the heterofermentative lactobacilli which have been shown to increase caries activity (Bowen, 1967). The results presented in this document clearly show that it does not enhance caries. To the contrary, it has a significant effect in reducing the risk of caries. Thus, LGG is suitable for use especially in preventive methods for combating caries and reducing caries risk.

The invention is described in greater detail with reference to the following examples, which are only intended to illustrate the invention and not to restrict its scope in any way.

### EXAMPLE 1.

### Production of Lactobacillus Rhamnosus LGG

LGG was cultivated in a medium comprising 5.0% of whey permeate (Valio Oy), 0.5% of casein hydrolysate (Valio Oy), 0.5% of technical yeast, and 0.0015% of MnSO₄×H₂O. The components of the medium were dissolved into water and the medium was sterilized (for 20 min at 120° C.). The cultivation was carried out at a temperature of 37° C. and a pH of 5.8 (adjusted with NH₄OH) for about 18 h and at a mixing rate of 100 rpm, After the cultivation the bacterial cells were concentrated, washed and freeze-dried using a 10% (v/v) protective agent supplement, such as a saccharose broth of 46%, or a similar alternative known to those skilled in the art. The final bacterial content was >1×10⁹ cfu/ml in the cultivation, >1×10¹⁰ cfu/g in the concentrate and >1×10¹¹ cfu/g in the freeze-dried powder.

### EXAMPLE 2

### Effects of Lactobacillus Rhamnosus LGG

Eighteen municipal day-care centers, situated in equal socio-economical areas in the city of Helsinki, Finland, participated in a randomized, double-blind, placebo-controlled intervention study which was designed to examine whether Lactobacillus rhamnosus GG has beneficial effects in the oral cavity, on the risk of caries and on caries in human subjects. 594 children, 1-6 years old, were included in the study.

The children drank either LGG milk (Gefilus®, Valio Ltd., Riihimäki Dairy, Finland; pasteurized cow's milk containing 1% fat and live Lactobacillus rhamnosus GG, ATCC 53103, bacteria 5-10¹⁰ CFU/ml) or control milk in the day-care centers 5 days a week for 7 months, from non-transparent color-coded milk containers. The control milk came from the same dairy but contained no added lactobacilli. The mean daily consumption of milk was 218 ml in the age group of 1- to 2-year-old children in the control group and 248 ml in the LGG group, 240 ml in controls and 232 ml in the LGG group in the age group of 3- to 4-year-old children, 269 ml and 257 ml respectively, in the age group of 5- to 6-year-old children. Apart from the 1- to 2-year-olds, no difference was observed in milk consumption between the groups.

The day-care personnel, parents, children and investigators were unaware of which milk contained the LGG strain throughout the study. The randomization code was not broken until the intention-to-treat analyses were performed. The day-care personnel every day recorded the volume of milk each child drank. The parents of the children kept daily symptom diaries and recorded the children's respiratory and gastrointestinal symptoms, infections diagnosed by doctors and possible antibiotic treatments. The use of other products containing Lactobacillus or other pharmaceutical lactic acid bacteria was forbidden for 4 weeks prior to and throughout the intervention. The use of fluoride varnish was forbidden during the study, but necessary dental treatment was allowed. Information about the social background, health attitudes and dental care habits of the children was obtained using structured questionnaires filled in by the parents at baseline. The interview was also repeated at the end of the study.

Experienced dentists in the Helsinki City Health Department examined the children's oral health according to the WHO criteria (WHO, Oral Health Surveys, Basic Methods, Geneva, World Health Organization, 1987). The same examiner carried out the examination of the same children at baseline and at the end, without reference to the baseline data.

In addition to the WHO guidelines, caries was recorded separately for occlusal, smooth (labial and oral) and approximal surfaces. The parameters studied were active caries (initial and decayed, dt/DT), cumulative caries (dmft/DMFT) and caries in occlusal, smooth and approximal surfaces.

Pooled plaque and saliva samples were taken by rotating a sterile cotton stick along the labial surfaces of the upper incisors, deciduous molars on the left buccal side under the prominence line of the teeth, the dorsal part of the tongue and at the bottom of the mouth, always in this order. The samples were taken in the day-care centers from all children always at the same time, i.e. 1 h after breakfast, at baseline, in the middle and at the end of the study. In addition, an unstimulated saliva sample was taken from the 5- to 6-year-old children with the free-flowing method. The pooled plaque and saliva samples were spread with cotton stick on Dentocult SM Strip mutans® slides (Orion Diagnostica, Espoo, Finland) and cultivated according to the manufacturer's instructions. The slides were counted under a stereomicroscope and scored as instructed by the manufacturer. The saliva samples were placed in Eppendorf tubes, chilled, centrifuged, deep-frozen and stored at −70 ⁵C until further analyses of salivary proteins.

Compliance to the LGG intervention was measured by assessing the faecal colonization with LGG using standard laboratory techniques. Samples from 100 children at baseline and from 60 children in the middle and at the end of the study were used.

Caries risk was determined on the basis of combined clinical and microbiological results comprising mutans streptococcus levels from dental plaque and saliva. Caries risk was classified into ‘high risk’ and ‘moderate risk’ groups. ‘High risk’ was recorded if the child had a dmft/DMFT or initial caries score >0 and a mutans streptococcus count over or equal to 10⁵ CFU/ml. ‘Moderate risk’ was recorded if either dmft/DMFT or initial caries score was >0 or the mutans streptococcus count was over of equal to 10⁵ CFU/ml. Caries risk was recorded as ‘low risk’ if no caries was detected and the mutans strep-tococcus count was below 10⁵ CFU/ml.

Logistic regression analyses were performed to compare the groups regarding caries and other binary response variables measured at the end of the study. The corresponding caries status at baseline was included as a grouping factor, because the children with increased caries risk factors at baseline developed more caries than those considered as ‘low risk’ children (Twetman et al., Use of the strip mutans in the assessment of caries in a group of preschool children, Int J Paediatr Dent 1994;4:245-250). Due to the minor differences between groups in age and sex distributions, age and sex were included as covariates when appropriate. The x² test was used for unadjusted analysis. In addition, age-stratified analyses were performed in order to control the possible confounding effect of age. The children were divided into three age groups: 1-2 years, 3-4 years and 5-6 years. All analyses were based on the intention-to-treat population. As the baseline-adjusted statistical analysis require complete data, the main results came from the children who participated in all study phases from baseline to the final examination. Statistical analyses were performed using the SPSS (Release 9.0) program.

The study protocol had been approved by the Ethical Committee of the Helsinki City Health Department. The parents gave their informed consent on behalf of the children.

Results on faecal colonization with LGG showed good compliance. At baseline, LGG was detected in 4% of the controls compared with 12% of the LGG group. The respective percentages at the end of the study were 15 versus 91%.

Mutans streptococcus levels of the pooled plaque and saliva samples from the different age groups are given in table 1. In the whole study population, the effects of LGG on caries were found to be positive, albeit not statistically significant. The baseline-adjusted odds ratios were: OR=0.80 (95% Cl 0.42-1.52) for dt/DT>0; OR=0.77 (0.40-1.46) for dmft/DMFT>0; OR=0.81 (0.47-1.40) for initial caries development; OR =0.72 (0.33-1.56) for approximal caries; OR=0.79 (0.43-1.45) for occlusal caries, and OR=0.61 (0.27-1.36) for smooth-surface caries development.

TABLE 1
Distribution of children with respect to different mutans steptococcus scores during the study

| | LGG group (n = 231) | | | | | | Control group (n = 230) | | | | | |
|---|---|---|---|---|---|---|---|---|---|---|---|---|
| | class 0, 1 | | class 2 | | class 3 | | class 0, 1 | | class 2 | | class 3 | |
| Study | n | % | n | % | n | % | n | % | n | % | n | % |

1- to 2-year-old children

| | | | | | | | | | | | | |
|---|---|---|---|---|---|---|---|---|---|---|---|---|
| Baseline | 26 | 83.9 | 4 | 12.9 | 1 | 3.2 | 34 | 89.5 | 4 | 10.5 | 0 | — |
| Middle | 28 | 90.3 | 0 | — | 3 | 9.7 | 36 | 94.7 | 2 | 5.3 | 0 | — |
| End | 29 | 93.5 | 2 | 6.5 | 0 | — | 32 | 84.2 | 5 | 13.2 | 1 | 2.6 |

3- to 4-year-old children

| | | | | | | | | | | | | |
|---|---|---|---|---|---|---|---|---|---|---|---|---|
| Baseline | 67 | 80.7 | 7 | 8.4 | 9 | 10.9 | 59 | 72.9 | 10 | 12.3 | 12 | 14.8 |
| Middle | 72 | 86.8 | 7 | 8.4 | 4 | 4.8 | 70 | 86.4 | 5 | 6.2 | 6 | 7.4 |
| End | 72 | 86.8 | 8 | 9.6 | 3 | 3.6 | 66 | 81.5 | 6 | 7.4 | 9 | 11.1 |

5- to 6-year-old children

| | | | | | | | | | | | | |
|---|---|---|---|---|---|---|---|---|---|---|---|---|
| Baseline | 82 | 70.1 | 15 | 12.8 | 20 | 17.1 | 78 | 77.2 | 16 | 15.9 | 7 | 6.9 |
| Middle | 96 | 82.1 | 11 | 9.4 | 10 | 8.5 | 89 | 88.1 | 7 | 6.9 | 5 | 5.0 |
| End | 96 | 82.1 | 10 | 8.5 | 11 | 9.4 | 80 | 79.2 | 16 | 15.8 | 5 | 5.0 |

The mutans steptococcus scores were assessed by using Dentocult SM Strip mutans: Class 0 and 1 = <100,000 CFU/ml; Class 2 = >100,000-<1,000,000 CFU/ml; Class 3 = ≧1,000,000 CFU/ml. 1- to 2-year-old children: LGG group, n = 31; controls, n = 38; 3- to 4-year-old children: LGG group, n = 83; controls, n = 81; 5- to 6-year-old children: LGG group, n = 117; control, n = 101.

The age-stratified dental caries results in the LGG and control groups are given in table 2. In the age group of the 3- to 4-year-old children, LGG milk appeared to protect against caries, especially in occlusal surfaces (OR=0.22, 95% Cl 0.04-1.06, p=0.059, baseline as a covariate). The base-line-adjusted odds ratios for the other caries variables used were: OR=0.34 (95% Cl 0.11-1.04) for dmft/DMFT

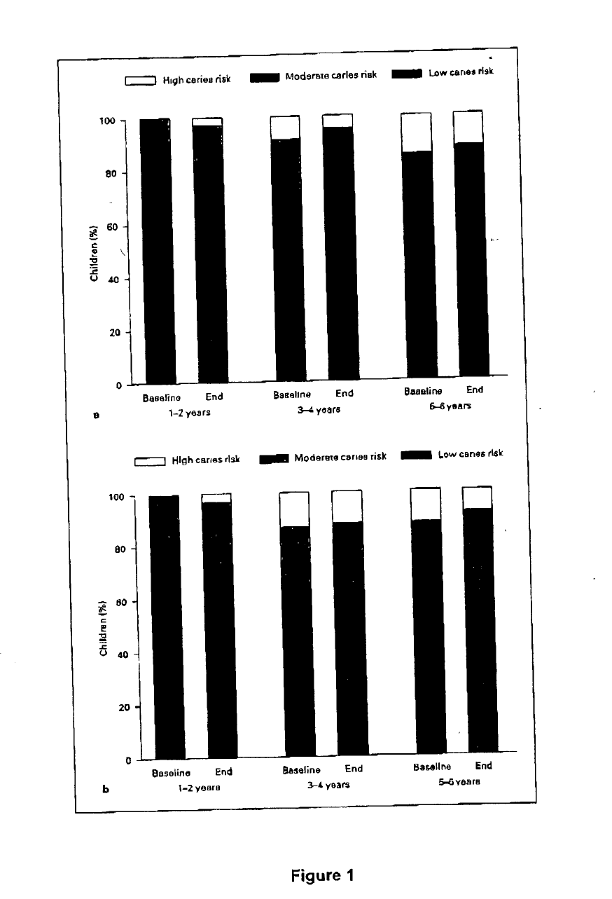

>0, and OR=0.34 (95% Cl 0.12-1.03) for dt/DT>0. In the LGG group, the calculated caries risk decreased from 40 to 34% in all age groups combined. In the control group, an increase was observed from 39 to 43%, respectively. Based on the results from the logistic regression analysis, the intervention with LGG was found to reduce the risk of caries statistically significantly: the baseline-adjusted odds ratio was 0.56 (95% Cl 0.36-0.88, p=0.01). Controlled for age and gender, the corresponding odds ratio was 0.51 (95% Cl 0.32-0.81, p=0.004). The caries risk figures are given in more detail in FIGS. 1a and b.

TABLE 2
Dental caries in the LGG and control groups at baseline and end of the study

| 1- to- 2-year-old children | | | | | 3- to- 4-year-old children | | | | | 5- to- 6-year-old children | | | | |
|---|---|---|---|---|---|---|---|---|---|---|---|---|---|---|
| LGG | | control | | | LGG | | control | | | LGG | | control | | |
| (n = 31) | | (n = 38) | | | (n = 83) | | (n = 81) | | | (n = 117) | | (n = 101) | | |
| n | % | n | % | p value | n | % | n | % | p value | n | % | n | % | p value |

dt/DT > 0

| | | | | | | | | | | | | | | | |
|---|---|---|---|---|---|---|---|---|---|---|---|---|---|---|---|
| Baseline | 0 | | 0 | | | 3 | 3.6 | 4 | 4.9 | | 10 | 8.5 | 10 | 9.9 | |
| End | 2 | 6.5 | 1 | 2.6 | 0.45 | 6 | 7.2 | 14 | 17 | 0.059 | 18 | 15 | 15 | 5 | 0.71 |
| dmft/DMFT > 0 | | | | | | | | | | | | | | | |
| Baseline | 0 | | 0 | | | 3 | 3.6 | 7 | 8.6 | | 16 | 14 | 17 | 17 | |
| End | 2 | 6.5 | 1 | 2.6 | 0.45 | 8 | 9.6 | 19 | 23 | 0.057 | 24 | 21 | 22 | 22 | 0.76 |
| Initial caries | | | | | | | | | | | | | | | |
| Baseline | 1 | 3.2 | 2 | 5.3 | | 18 | 22 | 21 | 26 | | 29 | 25 | 26 | 26 | |
| End | 2 | 6.5 | 4 | 10.5 | 0.64 | 17 | 20 | 23 | 28 | 0.30 | 33 | 28 | 30 | 30 | 0.85 |
| Approximal caries | | | | | | | | | | | | | | | |
| Baseline | 0 | | 0 | | | 2 | 2.4 | 8 | 9.9 | | 13 | 11 | 15 | 15 | |
| End | 0 | | 0 | | | 6 | 7.2 | 12 | 15 | 0.72 | 16 | 14 | 20 | 20 | 0.37 |
| Occlusal caries | | | | | | | | | | | | | | | |
| Baseline | 0 | | 2 | 5.3 | | 15 | 18 | 16 | 20 | | 20 | 17 | 20 | 20 | |
| End | 4 | 12.9 | 3 | 7.9 | 0.15 | 14 | 17 | 21 | 26 | 0.059 | 21 | 18 | 21 | 21 | 0.75 |
| Smooth surface caries | | | | | | | | | | | | | | | |
| Baseline | 1 | 3.2 | 0 | | | 8 | 9.6 | 7 | 8.6 | | 20 | 17 | 6 | 5.9 | |
| End | 0 | | 3 | 7.9 | 0.86 | 7 | 8.4 | 10 | 12 | 0.21 | 20 | 17 | 9 | 8.9 | 0.85 |

The results thus show less dental caries in the LGG group and lower mutans streptococcus counts at the end of the study. LGG was found to reduce the risk of caries significantly (OR=0.56, p=0.01; controlled for age and gender, OR=0.51, p 0.004). The effect was particularly clear in the 3- to 4-year-old. The results thus clearly show that Lactobacillus rhamnosus LGG has a beneficial effect on dental health and caries development.

1. Method for improving oral health in a subject, the method comprising administering to an individual in need of such treatment Lactobacillus rhamnosus GG, ATCC 53103, in an amount sufficient to achieve the desired result.

2. Method for improving dental health in a subject, the method comprising administering to an individual in need of such treatment Lactobacillus rhamnosus GG, ATCC 53103, in an amount sufficient to achieve the desired result.

3. Method for preventing and reducing the risk of caries in a subject, the method comprising administering to an individual in need of such treatment Lactobacillus rhamnosus GG, ATCC 53103, in an amount sufficient to achieve the desired result.

4. Method for improving oral health, improving dental health, and preventing and reducing the risk of caries in a subject, the method comprising administering to an individual in need of such treatment Lactobacillus rhamnosus GG, ATCC 53103, as such in an amount sufficient to achieve the desired result.

5. Method for improving oral health, improving dental health, and preventing and reducing the risk of caries in a subject, the method comprising administering to an individual in need of such treatment Lactobacillus rhamnosus GG, ATCC 53103, in a physiologically acceptable vehicle and in an amount sufficient to achieve the desired result.

6. Method according to claim 5, wherein the vehicle is milk or a milk product.

7. Method for improving oral health, improving dental health, or preventing and reducing the risk of caries in a subject, the method comprising administering to an individual in need of such treatment a calcium compound and Lactobacillus rhamnosus GG, ATCC 53103, in amounts sufficient to achieve the desired result.

8. Method according to claim 7, wherein the calcium compound is a soluble calcium salt such as calcium lactate, gluconate, phosphate, carbonate, citrate, malate, hydroxide, chloride or formate.
